(19) United States
(12) Patent Application Publication
(10) Pub. No.: US 2002/0193857 A1
(43) Pub. Date: Dec. 19, 2002

(54) Temperature retaining pad with removable cover

(76) Inventor: Kim D. Lavine, Grand Haven, MI (US)

Correspondence Address: PRICE HENEVELD COOPER DEWITT & LITTON, 695 KENMOOR, S.E., P O BOX 2567, GRAND RAPIDS, MI, 49501, US

(21) Appl. No.: 10/064,840

(22) Filed: Aug. 22, 2002

Related U.S. Application Data
(60) Provisional application No. 60/354,399, filed on Feb. 4, 2002.

(51) Int. Cl. A61F 7/00

(52) U.S. Cl. 607/114; 607/96

(57) ABSTRACT

A therapeutic device comprising an enclosed member and a cover. The enclosed member has a plurality of pieces of organic material therein. The cover encloses the enclosed member. The cover has a slit for the insertion and removal of the enclosed member. The pieces of organic material are capable of being exothermic when heated or endothermic when cooled. The enclosed member can be removed from the cover and the cover can be washed to clean the cover.

### CROSS REFERENCE TO RELATED APPLICATIONS

The present invention claims priority to Provisional Patent Application No. 60/354,399, filed on Feb. 4, 2002 entitled CORN BAG.

### BACKGROUND OF INVENTION

The field of endeavor to which this invention pertains is the therapeutic health and personal care field.

This product was created in response to a search for a natural, reusable alternative to heating pads and heatable silicone packs. This product was also developed in response to a need to provide consumers with a natural, low-cost alternative to heating pads and expensive heatable silicone packs. An additional goal of this product's development was to provide consumers with an attractive alternative to heating pads and heatable silicone packs.

Accordingly, a temperature retaining bag solving the aforementioned disadvantages and having the aforementioned advantages is desired.

### SUMMARY OF INVENTION

One aspect of the present invention is to provide a therapeutic device comprising an enclosed member and a cover. The enclosed member has a plurality of pieces of organic material therein. The cover encloses the enclosed member. The cover has a slit for the insertion and removal of the enclosed member. The pieces of organic material are capable of being exothermic when heated or endothermic when cooled. The enclosed member can be removed from the cover and the cover can be washed to clean the cover.

Another aspect of the present invention is to provide a method of administering heat comprising the steps of providing an enclosed member having pieces of exothermic organic material therein, and heating the enclosed member to heat the pieces of exothermic organic material therein. The method also includes the steps of placing the enclosed member within a cover by inserting the cover into a slit in the cover and applying the cover with the enclosed member therein to an area to be heated.

Yet another aspect of the present invention is to provide a method of administering cold comprising the steps of providing an enclosed member having pieces of endothermic organic material therein, and cooling the enclosed member to cool the pieces of endothermic organic material therein. The method also includes the steps of placing the enclosed member within a cover by inserting the cover into a slit in the cover and applying the cover with the enclosed member therein to an area to be cooled.

The object of the invention is to provide a new, unique and effective way to relieve pain and muscle soreness, to relieve tension, and to provide an overall sense of enjoyment and comfort, while also answering a need of consumers for an aesthetically attractive product.

I discovered that feed and/or seed corn, because of the density and weight of the kernel, was an excellent conductor of both heat and moisture, and that the corn kernel has a unique ability to retain and transmit both heat and moisture for long periods of time, up to two hours or more depending on environmental conditions, (e.g., under the blankets of a bed, or between a user's back and a leather chair). The corn bag can be reheated indefinitely without an appreciable loss of heat or moisture quality, and on average, costs significantly less. The uniqueness and novelty of my invention is significantly distinguished by the decorative, designer-quality fabrics that I encase the corn bags in. This added distinction of decorative, designer-quality fabric coverings distinguishes my invention significantly from aesthetically unattractive heating pads and silicone packs.

The feed and/or seed corn filled fabric pillow provides consumers with a unique and novel invention. It is unique and novel in its practical process application: the placement of feed corn in a bag for use as a transmitter of moist heat, for therapeutic relief of pain, muscle strain, tension relief, and for an overall sense of enjoyment and comfort. It is also unique and novel in its aesthetic application: a decorative covering of designer-quality cotton/flannel fabrics distinguishes it from aesthetically unattractive heating pads and silicone packs.

Advantages of the invention include a penetrating moist heat not found in heating pads or any silicone packs intended for application to the body for therapeutic use. The density of the corn kernel has a unique ability to retain and transmit both heat and moisture for an extended period of time; this advantage distinguishes corn pillows from heating pads and silicone packs intended for application to the body for therapeutic use.

The corn bag may be heated and taken to bed without the fear of electrical fires associated with heating pads, or possible burns, also associated with bed use of heating pads. The corn bag provides extended heat and therapeutic relief for up to two hours or more depending on environmental conditions. The corn bag provides an economical alternative to expensive silicone packs and heating pads. The corn bag, when encased in designer quality fabrics and presented as a wuvit®, provides consumers with an attractive alternative to aesthetically unappealing silicone packs and heating pads. The corn bag may be reused indefinitely without an appreciable loss of heat or moisture quality. The cost of use of the corn bag is significantly lower than the electrical costs associated with use of a heating pad.

These and other features, advantages, and objects of the present invention will be further understood and appreciated by those skilled in the art by reference to the following specification, claims and appended drawings.

### BRIEF DESCRIPTION OF DRAWINGS

FIG. 1 is a perspective exploded view of a therapeutic device of the present invention.

FIG. 2 is a perspective view of the therapeutic device of the present invention.

FIG. 3 is a perspective view of a second embodiment of the therapeutic device of the present invention.

FIG. 4 is a cross-section view of the second embodiment of the therapeutic device of the present invention.

FIG. 5 is a perspective view of a third embodiment of the therapeutic device of the present invention.

FIG. 6 is a flow diagram showing the method of using the therapeutic device to apply heat.

FIG. 7 is a flow diagram showing the method of using the therapeutic device to apply cold.

### DETAILED DESCRIPTION

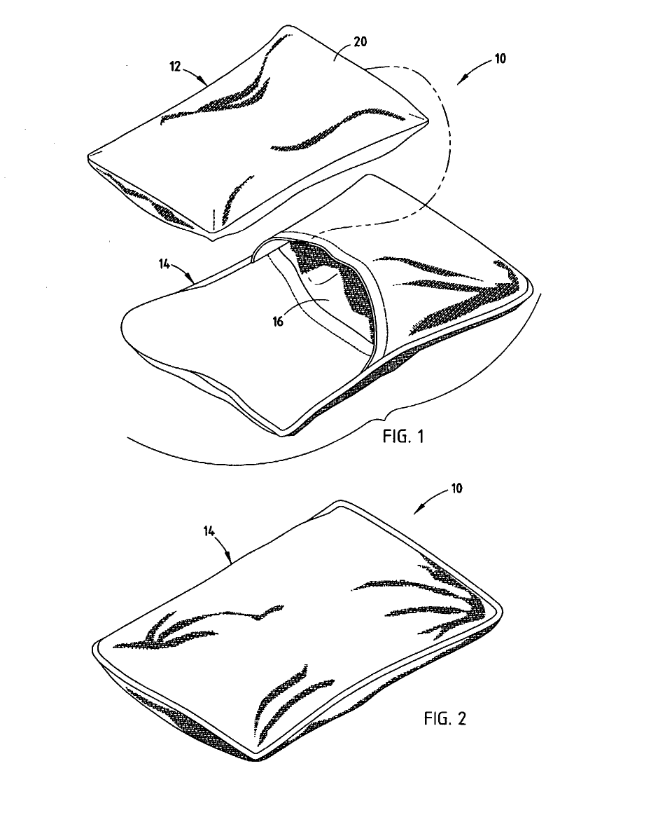

For purposes of description herein, the terms upper, lower, right, left, rear, front, vertical, horizontal, and derivatives thereof shall relate to the invention as orientated in FIG. 1. However, it is to be understood that the invention may assume various alternative orientations, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The reference number 10 (FIGS. 1 and 2) generally designates a therapeutic device comprising an enclosed member or bag 12 and a cover or pillowcase 14. The enclosed member 12 has a plurality of seeds, grains and/or fruit pits therein. The cover 14 encloses the enclosed member 12. The cover 14 has a slit 16 for the insertion and removal of the enclosed member 12. The seeds, grains and/or fruit pits are capable of being exothermic when heated or endothermic when cooled. The enclosed member 12 can be removed from the cover 14 and the cover 14 can be washed to clean the cover 14.

Preferably, the bag 12 is made of cloth 20 and filled with feed and/or seed corn. Feed corn is corn that has been dried for use as animal feed, e.g., cattle. Feed corn has been used primarily as an agricultural feed, and is available at most grain elevators and feed stores. Seed corn is corn kernels that have been dried, sorted and cleaned for the purpose of planting. The cloth bag is constructed with a sewing machine or serger, and once filled with corn, is sewn shut with a sewing machine or serger. The bag 12 can also be filled with rice, cherry pits or any other seeds, grains and/or fruit pits capable of being exothermic when heated or endothermic when cooled.

The exact dimension of the therapeutic device 10, as well as the amount and weight of feed and/or seed corn, varies, as different sized pillows 14 are made for different targeted areas on the body.

The therapeutic device 10 is used for the purpose of heating or freezing the corn filled bag 12 and applying it directly to the body for therapeutic use, for the purpose of relieving pain, reducing stress, relaxing muscles, reducing swelling, or providing overall general relaxation and enjoyment. The therapeutic device 10 provides a new, unique and effective way to relieve pain and muscle soreness, to relieve tension, and to provide an overall sense of warmth, enjoyment and comfort, achieved by heating or cooling the corn bag 10, and applying it directly to the body.

The therapeutic device 10 is used to apply heat by heating the bag 10 in a microwave oven for two to three minutes with proper rotation. The heating time may vary depending on the wattage of the microwave oven. The therapeutic device 10 should not be overheated and a light fingertip touch can be applied to the device 10 prior to full application to the skin to test the temperature of the device 10.

The device 10, currently for sale as a product named wuvit®, is marketed as a therapeutic health and personal care item. This invention is distinguished from other products by its use of seeds, grains and/or fruit pits as a heat conductor and transmitter, and the encasement of the bag 12 in a cloth pillowcase 14 for the direct therapeutic application of moist penetrating heat to the body. The moist heat that the seeds, grains and/or fruit pits emanate distinguishes it from other comparable therapeutic products, such as electric heating pads and silicone packs. The most unique and significant improvement corn bags possess over existing products is penetrating moist heat, a characteristic not found in heating pads or silicone packs. Seeds, grains and/or fruit pits, because of the density and weight of the unit, is an excellent conductor of both heat and moisture, and the seeds, grains and/or fruit pits have a unique ability to retain and transmit both heat and moisture for long periods of time. An improvement over existing products includes reduced purchase cost versus heating pads and silicone packs. Reduced usage cost is also a feature, when compared to electrical costs of using a heating pad.

An additional improvement over current products is that the device 10 may be heated and taken to bed without the fear of electrical fires associated with heating pads, or possible burns, also associated with bed use of heating pads. This is a significant benefit to those suffering from chronic pain or who are bedridden and must seek relief daily for long periods of time. It is also a benefit to those who may currently routinely fall asleep while using heating pads in bed, risking burns to the skin, or possible fire hazards.

The device 10 may be reused indefinitely without an appreciable loss of heat or moisture quality. The device 10 is also unique and novel in its aesthetic application: the decorative cover 14 of designer-quality cotton/flannel distinguishes it from unattractive heating pads and silicone packs.

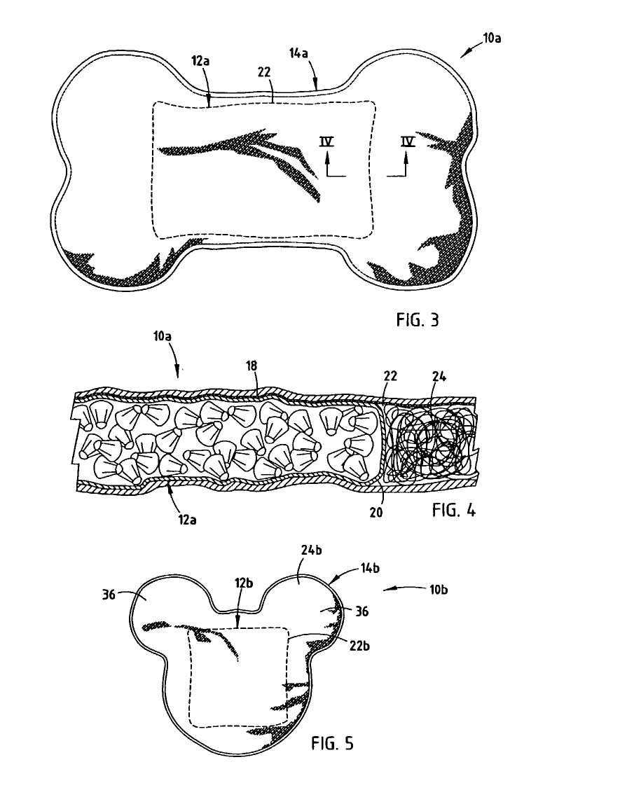

The reference numeral 10a (FIGS. 3-4) generally designates another embodiment of the present invention, having a second embodiment for the therapeutic device. Since the therapeutic device 10a is similar to the previously described therapeutic device 10, similar parts appearing in FIGS. 1-2 and FIGS. 3-4, respectively, are represented by the same, corresponding reference number, except for the suffix a in the numerals of the latter. The therapeutic device 10a includes the enclosed member or bag 12a as the first embodiment of the therapeutic device 10, but with a differently designed cover 14a. The cover 14a includes an internal washable plastic liner 18 located between the cloth 20a and the enclosed member 12a. The cover 12a of the therapeutic device 10a is dog-boned shaped and includes a rectangular center open portion 22 for accepting the enclosed member 12a and soft material 24 surrounding the rectangular center open portion 22 and defining the dog-bone shape of the cover 14a. The washable plastic liner 18 is located between the rectangular center open portion 22 and the soft material 24. The soft material 24 is preferably cotton or any other filler typically used in a pillow. The therapeutic device 10a can have shapes other than the dog-bone shape and is preferably used as a dog bed.

The reference numeral 10b (FIG. 5) generally designates another embodiment of the present invention, having a third embodiment for the therapeutic device. Since the therapeutic device 10b is similar to the previously described therapeutic device 10a, similar parts appearing in FIGS. 1-2 and FIG. 5, respectively, are represented by the same, corresponding reference number, except for the suffix b in the numerals of the latter. The therapeutic device 10b includes the enclosed member or bag 12a as the second embodiment of the therapeutic device 10a, but with a differently designed cover 14b. Preferably, the cover 14b is shaped like a head 34 and includes the rectangular center open portion 22b for accepting the enclosed member 12a and soft material 24b surrounding the rectangular center open portion 22b and defining the rest of the head 34, including ears 36.

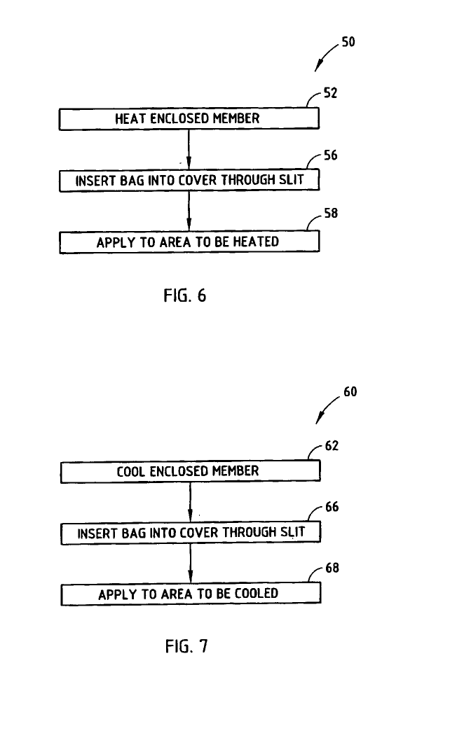

The illustrated therapeutic device 10 of the present invention relieves pain, reduces stress, relaxes muscles, reduces swelling, or provides overall general relaxation and enjoyment. Referring to FIG. 6, a method 50 of applying heat is shown. Beginning at step 52 of the method 50 of applying heat, the enclosed member 12 is heated, preferably in a microwave, to heat the plurality of exothermic seeds, grains and/or fruit pits therein. Thereafter, the enclosed member 12 is placed within the cover 14 by inserting the cover 14 into the slit 16 in the cover 14 at step 56. Finally, the cover 14 with the enclosed member 12 therein is applied to an area to be heated at step 58.

The illustrated therapeutic device 10 of the present invention relieves pain, reduces stress, relaxes muscles, reduces swelling, or provides overall general relaxation and enjoyment. Referring to FIG. 7, a method 60 of applying cold is shown. Beginning at step 62 of the method 60 of applying cold, the enclosed member 12 is cooled to cool the plurality of exothermic seeds, grains and/or fruit pits therein. Thereafter, the enclosed member 12 is placed within the cover 14 by inserting the cover 14 into the slit 16 in the cover 14 at step 66. Finally, the cover 14 with the enclosed member 12 therein is applied to an area to be cooled at step 68.

In the forgoing description, it will be readily appreciated by those skilled in the art that modifications may be made to the invention without departing from the concepts disclosed herein. Such modifications are to be considered as included in the following claims, unless these claims by their language expressly state otherwise.

1. A therapeutic device comprising:
an enclosed member having a plurality of pieces of organic material therein; and
a cover enclosing the enclosed member, the cover having a slit for the insertion and removal of the enclosed member;
wherein the organic material is capable of being exothermic when heated or endothermic when cooled; and
wherein the enclosed member can be removed from the cover and the cover can be washed to clean the cover.

2. The therapeutic device of claim 1, wherein:
the pieces of organic material comprise seeds, grains and/or fruit pits.

3. The therapeutic device of claim 2, wherein:
the plurality of seeds, grains and/or fruit pits comprises corn.

4. The therapeutic device of claim 2, wherein:
the plurality of seeds, grains and/or fruit pits comprises rice.

5. The therapeutic device of claim 2, wherein:
the plurality of seeds, grains and/or fruit pits comprises cherry pits.

6. The therapeutic device of claim 1, wherein:
the cover is comprised of cloth.

7. The therapeutic device of claim 6, wherein:
the cover further includes a washable plastic liner located between the cloth and the enclosed member.

8. The therapeutic device of claim 7, wherein:
the cover is dog-boned shaped and includes a rectangular center open portion for accepting the enclosed member and a soft material surrounding the rectangular center open portion and defining the dog-bone shape of the cover; and
the washable plastic liner is located between the rectangular center open portion and the soft material.

9. The therapeutic device of claim 6, wherein:
the cover includes a decorative design thereon.

10. The therapeutic device of claim 6, wherein:
the cover is head-shaped and includes a rectangular center open portion for accepting the enclosed member and a soft material surrounding the rectangular center open portion and defining the head.

11. A method of administering heat comprising:
providing an enclosed member having exothermic pieces of organic material therein;
heating the enclosed member to heat the exothermic pieces of organic material therein;
placing the enclosed member within a cover by inserting the cover into a slit in the cover; and
applying the cover with the enclosed member therein to an area to be heated.

12. The method of administering heat of claim 11, wherein:
the pieces of organic material comprise seeds, grains and/or fruit pits.

13. The method of administering heat of claim 12, wherein:
the plurality of exothermic seeds, grains and/or fruit pits comprises corn.

14. The method of administering heat of claim 12, wherein:
the plurality of exothermic seeds, grains and/or fruit pits comprises rice.

15. The method of administering heat of claim 12, wherein:
the plurality of exothermic seeds, grains and/or fruit pits comprises cherry pits.

16. The method of administering heat of claim 11, wherein:
the cover is comprised of cloth.

17. The method of administering heat of claim 16, further including:
providing the cover with a washable plastic liner for placement between the cloth and the enclosed member.

18. The method of administering heat of claim 17, wherein:
the cover is dog-boned shaped and includes a rectangular center open portion for accepting the enclosed member and a soft material surrounding the rectangular center open portion and defining the dog-bone shape of the cover; and
the washable plastic liner is located between the rectangular center open portion and the soft material.

19. The method of administering heat of claim 16, further including:
placing a decorative design on the cover.

20. The method of administering heat of claim 16, wherein:
the cover is head-shaped and includes a rectangular center open portion for accepting the enclosed member and a soft material surrounding the rectangular center open portion and defining the head.

21. The method of administering heat of claim 12, wherein:
the step of heating the enclosed member includes placing the enclosed member within a microwave and heating the enclosed member.

22. A method of administering cold comprising:
providing an enclosed member having pieces of endothermic organic material therein;
cooling the enclosed member to cool the pieces of endothermic organic material therein;
placing the enclosed member within a cover by inserting the cover into a slit in the cover; and
applying the cover with the enclosed member therein to an area to be cooled.

23. The method of administering heat of claim 22, wherein:
the pieces of organic material comprise seeds, grains and/or fruit pits.

24. The method of administering cold of claim 23, wherein:
the plurality of endothermic seeds, grains and/or fruit pits comprises corn.

25. The method of administering cold of claim 23, wherein:
the plurality of endothermic seeds, grains and/or fruit pits comprises rice.

26. The method of administering cold of claim 23, wherein:
the plurality of endothermic seeds, grains and/or fruit pits comprises cherry pits.

27. The method of administering cold of claim 23, wherein:
the cover is comprised of cloth.

28. The method of administering cold of claim 27, further including:
providing the cover with a washable plastic liner for placement between the cloth and the enclosed member.

29. The method of administering cold of claim 28, wherein:
the cover is dog-boned shaped and includes a rectangular center open portion for accepting the enclosed member and a soft material surrounding the rectangular center open portion and defining the dog-bone shape of the cover; and
the washable plastic liner is located between the rectangular center open portion and the soft material.

30. The method of administering cold of claim 27, further including:
placing a decorative design on the cover.

31. The method of administering cold of claim 27, wherein:
the cover is head-shaped and includes a rectangular center open portion for accepting the enclosed member and a soft material surrounding the rectangular center open portion and defining the head.

32. The method of administering cold of claim 23, wherein:
the step of cooling the enclosed member includes placing the enclosed member within an area to be cooled and cooling the enclosed member.
